(19) United States
(12) Patent Application Publication
(10) Pub. No.: US 2019/0001083 A1
(43) Pub. Date: Jan. 3, 2019

(54) AEROSOL GENERATION WITH REDUCED SOUND GENERATION

(71) Applicant: KONINKLIJKE PHILIPS N.V., EINDHOVEN (NL)

(72) Inventor: MARKUS HIJLKEMA, CHICHESTER (GB)

(21) Appl. No.: 16/065,161

(22) PCT Filed: Dec. 19, 2016
(86) PCT No.: PCT/EP2016/081633

§ 371 Date: Jun. 22, 2018

(30) Foreign Application Priority Data
Dec. 23, 2015 (EP) 15202444.4

(51) Int. Cl. A61M 15/00; A61M 11/00

(52) U.S. Cl. CPC A61M 15/009; A61M 2205/42; A61M 11/003; A61M 15/008

(57) ABSTRACT

The present invention relates to generating aerosols. In order to provide an improved aerosol generation with enhanced user friendliness, an aerosol generator (10) is provided that comprises an aperture structure (12) comprising a plurality of apertures, a transducer (14), a reservoir space (16) for receiving a liquid, from which liquid droplets are to be generated in order to produce an aerosol, and a control unit (18). The reservoir space is arranged adjacent to the aperture structure. The transducer is configured to generate a vibrational movement to be transferred as a relative movement between the liquid and the aperture structure. The control unit is configured to activate the transducer to vibrate for a first period having a first time to generate a relative movement between a liquid and the aperture structure, the movement being in a transverse direction to the aperture structure to urge liquid through the plurality of apertures to produce a plurality of droplets; and to pause the transducer for a second period having a second time; and to continuously repeat the activation and pause in an alternating manner. The control unit is further configured to randomly vary at least the second time for a following repeat loop at least after a determined maximum number of repetitions.

### FIELD OF THE INVENTION

The present invention relates to generating aerosols, and relates in particular to an aerosol generator, a nebulizer system and a method for operating a nebulizer, as well as to a computer program element and a computer-readable medium.

### BACKGROUND OF THE INVENTION

For administration of medicines for example to the lungs, inhaling of an aerosol maybe provided. For example, an aerosol of the particular medicine can be produced by a nebulizer to enable the deposition of medicine in the lungs. As an example, WO 2013/072863 A1 describes a nebulizer and the generation of an aerosol by operating an actuator in a pulsed operation mode. However, the generation of aerosol may be accompanied by the generation of noise, which may affect user acceptance.

### SUMMARY OF THE INVENTION

There may thus be a need to provide an improved aerosol generation with enhanced user friendliness.

The object of the present invention is solved by the subject-matter of the independent claims, wherein further embodiments are incorporated in the dependent claims.

According to the present invention, an aerosol generator is provided comprising an aperture structure comprising a plurality of apertures. The aerosol generator further comprises a transducer and a reservoir space for receiving a liquid, from which liquid droplets are to be generated in order to produce an aerosol. Further, a control unit is also provided. The reservoir space is arranged adjacent to the aperture structure. The transducer is configured to generate a vibrational movement to be transferred as a relative movement between the liquid and the aperture structure. The control unit is configured to activate the transducer to vibrate for a first period having a first time to generate a relative movement between a liquid and the aperture structure, the movement being in a transverse direction to the aperture structure to urge liquid through the plurality of apertures to produce a plurality of droplets. The control unit is further configured to pause the transducer for a second period having a second time. The control unit is further configured to continuously repeat the activation and pause in an alternating manner. The control unit is further configured to randomly vary at least the second time for a following repeat loop at least after a determined maximum number of repetitions.

As a result, i.e. due to the random variation of at least the second time, the generation of an aerosol is accompanied with a more or less undefined noise, instead of a certain frequency that might be perceived as annoying to a user. The variation of the at least second time is provided in order to achieve a mode of operation of, for example, a nebulizer, which is perceived as less noticeable to a user. Although some noise is still produced, the mode may be referred to as silent mode.

The term “transverse” relates to a movement transverse to the surface of the aperture structure, for example in a perpendicular manner. Due to the transverse movement, e.g. liquid droplets are generated by a mesh structure.

According to an example, the aperture structure is provided as at least one of the group of a mesh structure with a plurality of connected strands forming openings, a net or web with a plurality of net-members and apertures in-between, and a nozzle plate with a base structure having a plurality of nozzles.

According to an example, the transducer is a piezo element.

According to an example, the control unit is a microcontroller provided with a counting module and a random number generator. The counting module is configured to count pulses and to toggle between an on-state and an off-state. The random number generator is configured to generate a new pulse time.

According to an example, the control unit is configured to activate the transducer to supply the droplets in a burst mode, in which a number of subsequent droplets pluralities are provided in the first period.

According to the invention, also a nebulizer system is provided comprising an air-flow path with an air inlet and an air outlet. Further, an aerosol generator according to one of the preceding examples is provided. The aerosol generator is arranged in fluid communication with the air-flow path to supply the plurality of droplets to an air stream to produce an aerosol.

According the invention, also a method for operating a nebulizer is provided that comprises the following steps:

a) Activating a transducer to vibrate for a first period having a first time to generate a relative movement between a liquid and an aperture structure, the movement being in a transverse direction to the aperture structure.
b) Pausing the transducer for a second period having a second time.

Steps a) and b) are continuously repeated in an alternating manner. For the repeating, a maximum number of repetitions is determined after which at least the second time is randomly varying for a following repeat loop.

According to an example, the second time is randomly chosen from within a predetermined range.

According to an example, the first time is constant for the repeat loop.

According to another example, the first time is randomly varying for the repeat loop, wherein the variation of the first time is centered around a predetermined constant time.

According to an aspect, a mode of operation is provided, in which a transducer is activated for a first time and is then hold to pause for a second time, but the second time is varied for consecutive steps in order to avoid a determined frequency of the aerosol generation.

These and other aspects of the present invention will become apparent from and be elucidated with reference to the embodiments described hereinafter.

### BRIEF DESCRIPTION OF THE DRAWINGS

Exemplary embodiments of the invention will be described in the following with reference to the following drawings:

FIG. 1 schematically shows a setup of an example of an aerosol generator;

FIG. 2 schematically shows three different examples of an aerosol generator;

FIG. 3 schematically shows an example of a nebulizer system;

FIG. 4 schematically shows an example of a method for operating a nebulizer; and

FIG. 5 shows a further diagram for the operation of the nebulizer.

### DETAILED DESCRIPTION OF EMBODIMENTS

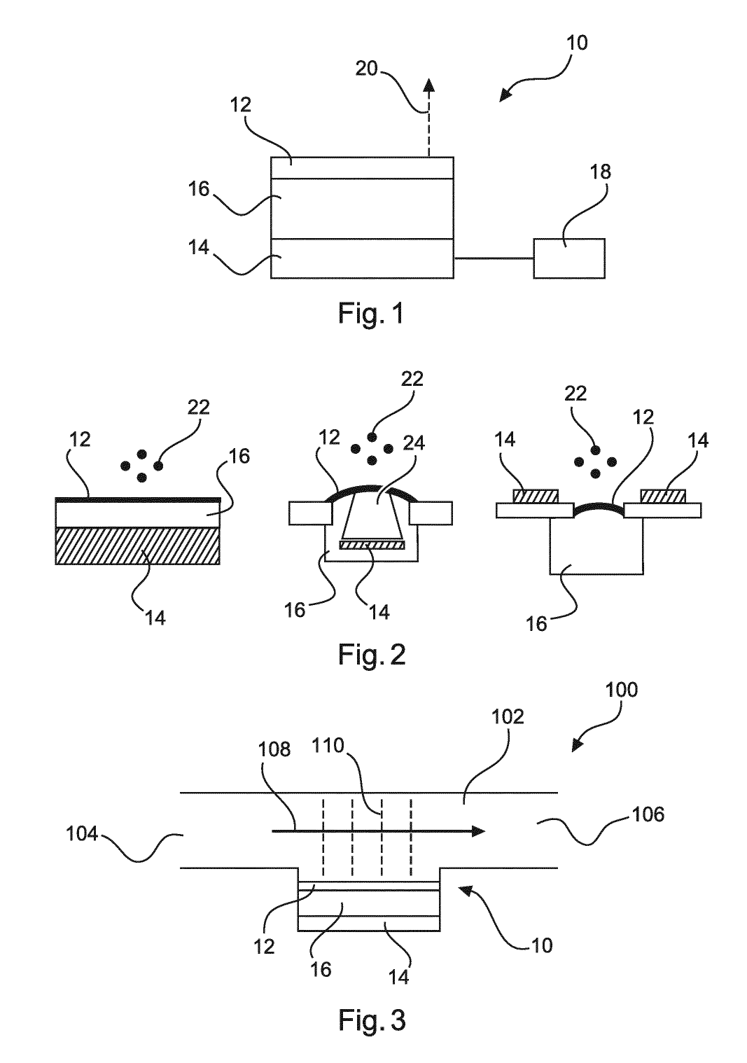

FIG. 1 shows an aerosol generator 10 comprising an aperture structure 12 with a plurality of apertures (not shown in detail). Further, a transducer 14 is provided and a reservoir space 16 for receiving a liquid, from which liquid droplets are to be generated in order to produce an aerosol. Further, a control unit 18 is provided. The reservoir space 16 is arranged adjacent to the aperture structure 12, for example between the transducer 14 and the aperture structure 12. Further, the transducer 14 is configured to generate a vibrational movement to be transferred as a relative movement between the liquid (in the reservoir space 16) and the aperture structure 12. The control unit 18 is configured to activate the transducer 14 to vibrate for a first period having a first time to generate a relative movement between a liquid and the aperture structure 12, the movement being in a transverse direction to the aperture structure 12 to urge a liquid through the plurality of apertures to produce a plurality of droplets, as indicated with dashed arrow 20. The control unit 18 is further configured to pause the transducer 14 for a second period having a second time and continuously repeat the activation in pause in an alternating manner. The control unit 18 is further configured to randomly vary at least the second time for a following repeat loop at least after a determination maximum number of repetitions.

In an example, the variation of the second time is centered around a predetermined constant time.

In an example, the liquid is arranged in a tank volume (not shown) and the tank volume is fluidly connected to the reservoir space 16 for receiving the liquid.

In an example, the reservoir space for receiving the liquid is a fluid reservoir provided to accommodate the liquid.

According to an example, not shown in detail, the aperture structure 12 is provided as at least one of the group of a mesh structure with a plurality of connected strands forming openings, a net or web with a plurality of net-members and apertures in-between, and a nozzle plate with a base structure having a plurality of nozzles.

In an example, the base structure is plate-liked formed. In an example, the reservoir space is a cavity.

FIG. 2 schematically shows three examples of an aerosol generator geometry. In the left part, the transducer 14 is provided below the reservoir space 16, on top of which the aperture structure 12, for example an aperture plate, is provided. Droplets 22 are schematically indicated.

In the middle part, a further example is shown, in which the transducer 14 is provided in combination with a further coupling element 24 to provide the respective relative movement such that the aperture plate 12 provides the droplets 22.

In the right part, the transducer 14 is arranged attached to a base-like structure, to which the aperture plate 12 is attached, for example in a dome-like manner. The reservoir space 16 is provided below.

According to an example, the transducer 14 is provided with a plate-like transducer structure (see for example FIG. 2, left part) for transferring the movement to the liquid in the reservoir space 16. The aperture structure 12 is provided as a plate-like structure comprising the plurality of apertures. Preferably, the plate-like transducer structure and the plate-like aperture structure are arranged displaced in a parallel manner.

In an example, the aerosol generator is a flat plate aerosol generator.

In an example, the transducer is a piezo element.

In an example, not further shown, the control unit 18 is a microcontroller provided with a counting module and a random number generator. The counting module counts pulses and toggles between an on-state and an off-state. The random number generator generates a new pulse time.

In an example, the reservoir space is arranged such that at least the transducer 14 is in contact with the liquid.

In an example, the reservoir space is arranged such that the mesh structure is in contact with the liquid.

In an example, not further shown, the control unit 18 is configured to activate the transducer to supply the droplets in a burst mode (see below), in which a number of subsequent droplets pluralities are provided in the first period.

In an example, a feedback arrangement may be provided in order to control absence of audible sound by adaptation of the random variation.

For example, the variation may be based on an equation, and upon detection of audible sound above a threshold, the equation is amended or adapted.

FIG. 3 shows a nebulizer system 100 comprising an air-flow path 102 with an air inlet 104 and an air outlet 106. Further, an aerosol generator 10 according to one of the preceding examples is provided. An arrow 108 indicates an air-flow and droplets 110 are indicated produced by the aerosol generator 10.

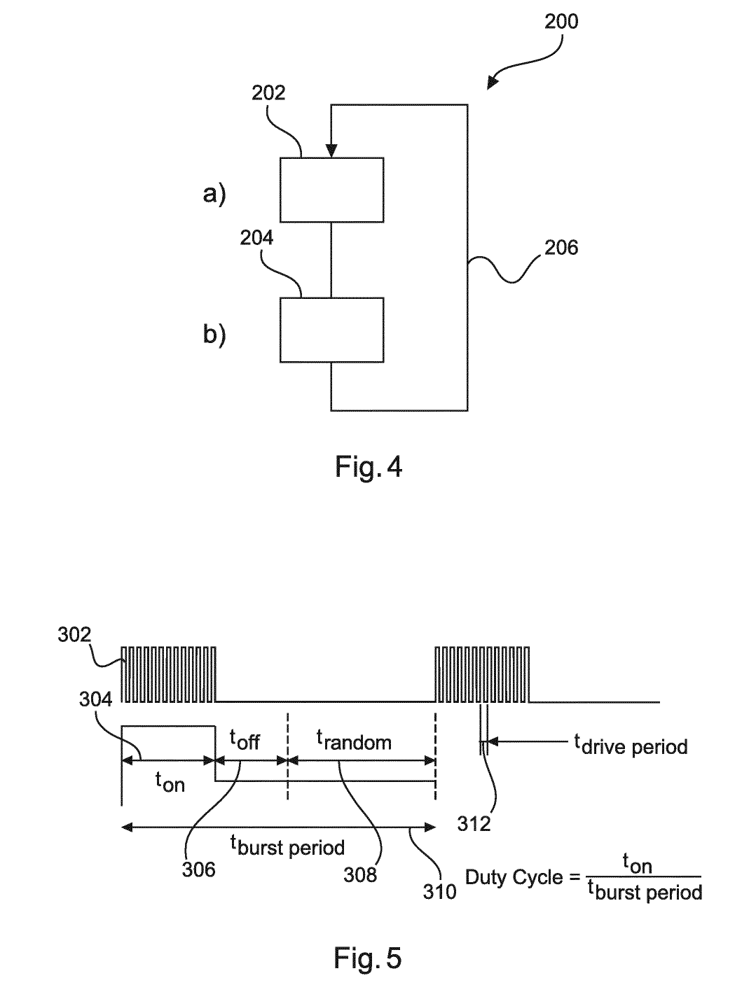

FIG. 4 shows a method 200 for operating a nebulizer, comprising the following steps: In a first step 202, also referred to as step a), a transducer is activated to vibrate for a first period having a first time to generate a relative movement between a liquid and an aperture structure, the movement being in a transverse direction to the aperture structure. In a second step 204, also referred to as step b), the transducer is paused for a second period having a second time. Further, steps a) and b) are continuously repeated in an alternating manner as indicated with repeat arrow loop 206. For the repeating, a maximum number of repetitions is determined, after which at least the second time is randomly varying for a following repeat loop.

In an example, the variation of the second time is centered around a predetermined constant time. The “continuously repeated” refers to a consecutive repetition of the activation and the holding of the transducer. The steps are repeated in a sort of repeat loop.

The term “to vibrate” relates to an oscillating movement of the transducer. The term “pausing” relates to deactivating the transducer, or holding or stopping the transducer to oscillate or vibrate, or decreasing significantly the intensity of oscillation or vibration.

In a further example, the second time is randomly chosen from within a predetermined range.

In an example, the randomly chosen second time varies in a random fashion but provides a predetermined average value for the second time. In an example, the first time is a pulse-on time in which the transducer generates a pulsating movement. The second time is a pulse-off time in which the transducer does not generate a pulsation movement.

In an example, the second time is varied between approximately 1 and 1800 microseconds (μs), but the average time is approximately 900 microseconds.

In an example, the average cycle time is 1000 microseconds. A resulting frequency is thus 1 kHz regarding the average value. However, due to randomly varying, a white noise is generated instead of a 1 kHz tone.

It must be noted that the examples that are given are provided as examples for explaining the particular approach in achieving an improved acoustic appearance. Of course, other variations are also provided.

For an example, t random=900, with an activation time t on=100; and hence t burst period=1000. In other words, this results in 100+900=1000.

In an example, the first time is constant for the repeat loop. In another example, the first time is approximately 100 microseconds.

The first time may also be randomly varying for the repeat loop, and the variation of the first time is centered around a predetermined constant time.

In another example, a counting is provided for determining the duration of the second time, and for the second period a random number is generated, wherein for consecutive second periods different numbers are generated.

In FIG. 5, a diagram is indicated showing a first part, in which pulses 302 are shown to drive the circuit. This is also referred to as the activation time tₒₙ, indicated with an arrow 304. The tₒₙ time is followed by a deactivated time t_{off}, indicated with double arrow 306. However, a following off-time is provided with a random length, also referred to as t_{random} indicated with double arrow 308. The tₒₙ time, the t_{off} time and the t_{random} time are providing a t_{burst period} time, as indicated with double arrow 310. A further arrow 312 indicates the actual drive period from the individual pulses, which drive period is also indicated with t_{drive period}.

As a resulting duty cycle, the tₒₙ time is divided by the t_{burst period} time.

In an example, the fixed t_{off} time could be zero, but may also vary. As an example, a tₒₙ time of 50 to 500 μs is provided, and a t_{off} time with 100 μs, but also preferably smaller. For the t_{random} time it is noted that this time may be based on a clock period times the average value of an n-bit random number. For instance, if an 8 bit random number is generated, the average number will be 2⁸⁻¹=128, and t_{random} will be 128 times the clock period. Hence, the following can be provided:

Clock period 5.33 μs

0-bit=No random number (0).

Mode 1: 7-bit=a random number of between 1 and 128 (yields 5.33 μs to 682.66 μs)

Mode 2: 8-bit=a random number of between 1 and 256 (yields 5.33 μs to 1.3653 ms)

Mode 3: 9-bit=a random number of between 1 and 512 (yields 5.33 μs to 2.7306 ms)

Mode 4: 10-bit=a random number of between 1 and 1024 (yields 5.33 μsec to 5.4613 ms)

Mode 5: 11-bit=a random number of between 1 and 2048 (yields 5.33 μs to 10.922 ms)

t_{drive} time, typically 500 ns-10 μs, is provided.

It is further noted that for driving a generator in a burst mode, this can be provided to reduce the average current to improve the lifetime of a transducer when driving a certain amplitude required for droplet formation. Further, it may also be provided to clear liquid from the aperture plate surface, in particular for low surface tension liquids. Further, the burst mode can be used to control the size of the droplets, as with longer tₒₙ times, droplet size may be increased.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid-state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to a aerosol generator whereas other embodiments are described with reference to the nebulizer system and the method for operating a nebulizer. However, a person skilled in the art will gather from the above that, unless otherwise notified, in addition to any combination of features belonging to one subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments.

Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An aerosol generator comprising:
an aperture structure comprising a plurality of apertures;
a transducer;
a reservoir space for receiving a liquid, from which liquid droplets are to be generated in order to produce an aerosol; and
a control unit;
wherein the reservoir space is arranged adjacent to the aperture structure; and wherein the transducer is configured to generate a vibrational movement to be transferred as a relative movement between the liquid and the aperture structure; wherein the control unit is configured to:
activate the transducer to vibrate for a first period having a first time to generate a relative movement between a liquid and the aperture structure, the movement being in a transverse direction to the aperture structure to urge liquid through the plurality of apertures to produce a plurality of droplets;
pause the transducer for a second period having a second time;
continuously repeat the activation and pause in an alternating manner; and
wherein the control unit is further configured to randomly vary at least the second time for a following repeat loop at least after a determined maximum number of repetitions.

2. Aerosol generator according to claim 1, wherein the aperture structure is provided as at least one of the group of:
a mesh structure with a plurality of connected strands forming openings;
a net or web with a plurality of net-members and apertures in-between; and
a nozzle plate with a base structure having a plurality of nozzles.

3. Aerosol generator according to claim 1, wherein the transducer is provided with a plate-like transducer structure for transferring the movement to the liquid in the reservoir space; and wherein the aperture structure is provided as a plate-like structure comprising the plurality of apertures; and wherein, preferably, the plate-like transducer structure and the plate-like aperture structure are arranged displaced in a parallel manner.

4. Aerosol generator according to claim 1, wherein the transducer is a piezo element.

5. Aerosol generator according to claim 1, wherein the control unit is a microcontroller provided with a counting module and a random number generator; wherein the counting module is configured to count pulses and to toggle between an on-state and an off-state; and wherein the random number generator is configured to generate a new pulse time.

6. Aerosol generator according to claim 1, wherein the control unit is configured to activate the transducer to supply the droplets in a burst mode, in which a number of subsequent droplets pluralities are provided in the first period.

7. Aerosol generator according to claim 1, wherein a feedback arrangement is provided in order to control absence of audible sound by adaptation of the random variation.

8. A nebulizer system comprising:
(a) an air-flow path with an air inlet and an air outlet; and
(b) an aerosol generator comprising:
comprising
(1) an aperture structure comprising a plurality of apertures;
(2) a transducer;
(3) a reservoir space for receiving a liquid, from which liquid droplets are to be generated in order to produce an aerosol; and
(4) a control unit, wherein the reservoir space is arranged adjacent to the aperture structure; and wherein the transducer Is configured to generate a vibrational movement to be transferred as a relative movement between the liquid and the aperture structure; wherein the control unit is configured to:
(i) activate the transducer to vibrate for a first period having a first time to generate a relative movement between a liquid and the aperture structure, the movement being in a transverse direction to the aperture structure to urge liquid through the plurality of apertures to produce a plurality of droplets,
(ii) pause the transducer for a second period having a second time; and
(iii) continuously repeat the activation and pause in an alternating manner, wherein the control unit is further configured to randomly vary at least the second time for a following repeat loop at least after a determined maximum number of repetitions; wherein the aerosol generator is arranged in fluid communication with the air-flow path to supply the plurality of droplets to an air stream to produce an aerosol.

9. A method for operating a nebulizer, comprising the following steps:
a) activating a transducer to vibrate for a first period having a first time to generate a relative movement between a liquid and an aperture structure, the movement being in a transverse direction to the aperture structure; and
b) pausing the transducer for a second period having a second time; wherein steps a) and b) are continuously repeated in an alternating manner; and wherein, for the repeating, a maximum number of repetitions is determined after which at least the second time is randomly varying for a following repeat loop.

10. A method according to claim 9, wherein the second time is randomly chosen from within a predetermined range.

11. A method according to claim 9, wherein the first time is constant for the repeat loop.

13. A method according to claim 9, wherein the first time is randomly varying for the repeat loop, wherein the variation of the first time is centered around a predetermined constant time.

13. Method according to claim 9, wherein a counting is provided for determining the duration of the second time; and wherein for the second period a random number is generated, wherein for consecutive second periods different numbers are generated.

14. A computer program element for controlling an apparatus according to the following method:
a) activating a transducer to vibrate for a first period having a first time to generate a relative movement between a liquid and an aperture structure, the movement being in a transverse direction to the aperture structure; and
b) pausing the transducer for a second period having a second time, wherein steps a) and b) are continuously repeated in an alternating manner, and wherein, for the repeating, a maximum number of repetitions is determined after which at least the second time is randomly varying for a following repeat loop.

15. (canceled)
